(19) United States
(12) Patent Application Publication
(10) Pub. No.: US 2026/0365212 A1
(43) Pub. Date: Nov. 19, 2026

(54) WORK SURFACE FOR A UTILITY CART

(71) Applicant: Apex Brands, Inc., Apex, NC (US)

(72) Inventors: Jesse HIGGINS, Raleigh, NC (US); Robert WARREN, Charlotte, NC (US); Zhihong FU, Cary, NC (US); Kyle ROMANICK, Fuquay-Varina, NC (US)

(21) Appl. No.: 19/137,068

(22) PCT Filed: Dec. 4, 2023
(86) PCT No.: PCT/US2023/082259

§ 371 Date: Jun. 9, 2025

Related U.S. Application Data
(60) Provisional application No. 63/431,845, filed on Dec. 12, 2022.
(60) Provisional application No. 63/468,565, filed on May 24, 2023.

(51) Int. Cl. B62B 3/02; B62B 5/06

(52) U.S. Cl. CPC B62B 3/02; B62B 5/06

(57) ABSTRACT

A utility cart may include a handle, a top support platform, to which the handle may be operably coupled, a bottom support platform which may be parallel to the top support platform, at least one upright support member which may operably couple the bottom support platform to the top support platform, a mobility assembly which may be operably coupled to the bottom support platform, and a work surface that may be alternatingly removably operably coupled to the top support platform via a first support assembly and a second support assembly. The first support assembly and the second support assembly may operably couple the work surface to the top support platform in different orientations.

### TECHNICAL FIELD

Example embodiments generally relate to utility carts and, in particular, relate to utility carts that are provided with a work surface for improved utility and versatility.

### BACKGROUND

Utility carts may be commonly used in various settings ranging from dedicated workspaces such as labs, garages, and warehouses or other storage facilities to miscellaneous settings such as in offices, homes, restaurants, and many more. Within the various settings that utility carts may be used, utility carts may also be used for various purposes including but not limited to transporting objects, storing objects, working on objects, displaying objects, and many more. Utility carts may essentially be ubiquitously used in any sort of productive setting involving objects that may be desirable to transport due to their widely applicable and often simple design allowing the cart to be a useful resource in a large amount of use cases.

Existing utility carts may be rather simplistic and outdated in their design considering how they have come to be commonly used today. In this regard, users of utility carts may be using utility carts for more, and different, tasks than in the past. As such, there may be a need to develop an improved design for a utility cart that may be better suited for the increasingly wide variety of tasks for which utility carts may be utilized.

### BRIEF SUMMARY OF SOME EXAMPLES

Some example embodiments may provide for a utility cart. The utility cart may include a handle, a top support platform, to which the handle may be operably coupled, a bottom support platform which may be parallel to the top support platform, at least one upright support member which may operably couple the bottom support platform to the top support platform, a mobility assembly which may be operably coupled to the bottom support platform, and a work surface that may be alternatingly removably operably coupled to the top support platform via a first support assembly and a second support assembly. The first support assembly and the second support assembly may operably couple the work surface to the top support platform in different orientations.

Some example embodiments may provide for an accessory support assembly for a utility cart. The accessory support assembly may include a handle, a top support platform to which the handle may be operably coupled, the top support platform may include a plurality of accessory slots which may be disposed around a perimeter thereof, at least one extension arm which may operably couple the handle to the top support platform, and a work surface that may be alternatingly removably operably coupled to the top support platform via a first support assembly and a second support assembly. The first support assembly and the second support assembly may operably couple the work surface to the top support platform in different orientations.

Some example embodiments may provide for a work surface for a utility cart. The work surface may include a planar member which may have a larger area than a top support platform of the utility cart, and a prop rod which may be disposed on an underside of the planar member. The work surface may be alternatingly removably operably coupled to the top support platform via a first support assembly and a second support assembly. The first support assembly may be a hinge assembly which may operably couple the planar member to the top support platform and may define a pivot axis there through. The second support assembly may be a hook assembly which may operably couple the planar member to the top support platform at an opposite edge of the planar member from the hinge assembly.

### BRIEF DESCRIPTION OF THE SEVERAL VIEWS OF THE DRAWING(S)

Having thus described some example embodiments in general terms, reference will now be made to the accompanying drawings, which are not necessarily drawn to scale, and wherein:

FIG. 1 illustrates a perspective view of a utility cart according to an example embodiment;

FIG. 2 illustrates a perspective view of the utility cart having a work surface in a first position according to an example embodiment;

FIG. 3 illustrates a front view of the utility cart having the work surface in the first position according to an example embodiment;

FIG. 4 illustrates a side view of the utility cart having the work surface in the first position according to an example embodiment;

FIG. 5 illustrates a perspective view of the utility cart having the work surface in a second position according to an example embodiment;

FIG. 6 illustrates a front view of the utility cart having the work surface in the second position in accordance with an example embodiment;

FIG. 7 illustrates a perspective view of the utility cart having the work surface in a third position in accordance with an example embodiment;

FIG. 8 illustrates a front view of the utility cart having the work surface in the third position in accordance with an example embodiment;

FIG. 9 illustrates a side view of the utility cart having the work surface in the third position in accordance with an example embodiment;

FIG. 10 illustrates a perspective exploded view of the utility cart and the work surface in accordance with an example embodiment;

FIG. 11 illustrates a front exploded view of the utility cart and the work surface in accordance with an example embodiment;

FIG. 12 illustrates a side exploded view of the utility cart and the work surface in accordance with an example embodiment;

FIG. 13 illustrates an isolated perspective view of the work surface in accordance with an example embodiment;

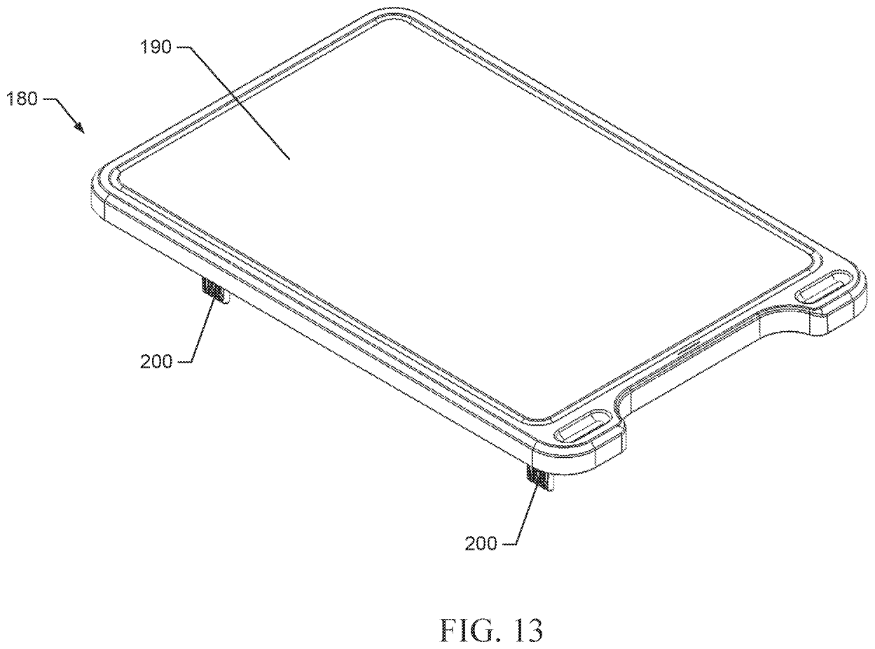

FIG. 14 illustrates an isolated bottom view of the work surface in accordance with an example embodiment;

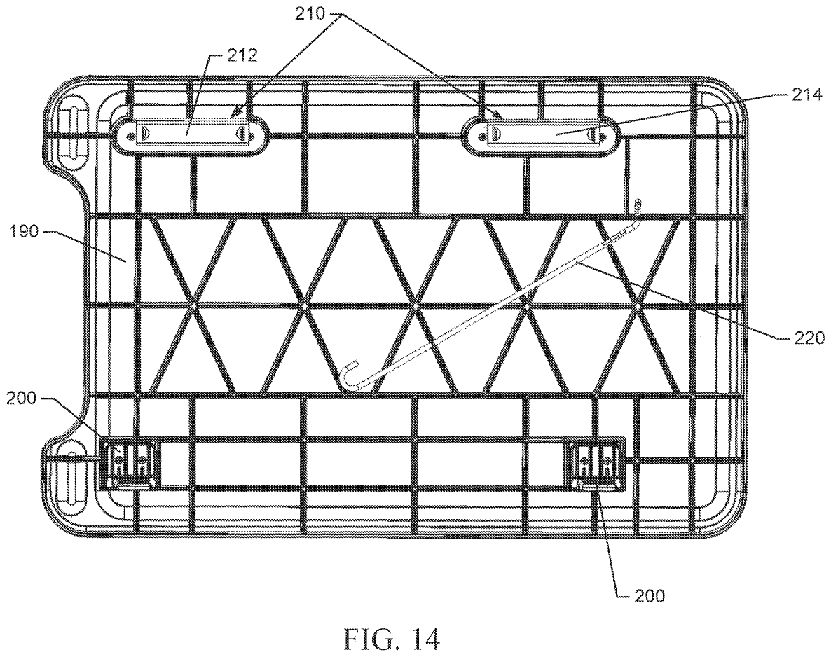

FIG. 15 illustrates a close up perspective view of the hinge assembly in accordance with an example embodiment; and

FIG. 16 illustrates a close up perspective view of the hook assembly in accordance with an example embodiment.

### DETAILED DESCRIPTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout. Furthermore, as used herein, the term “or” is to be interpreted as a logical operator that results in true whenever one or more of its operands are true. As used herein, operable coupling should be understood to relate to direct or indirect connection that, in either case, enables functional interconnection of components that are operably coupled to each other.

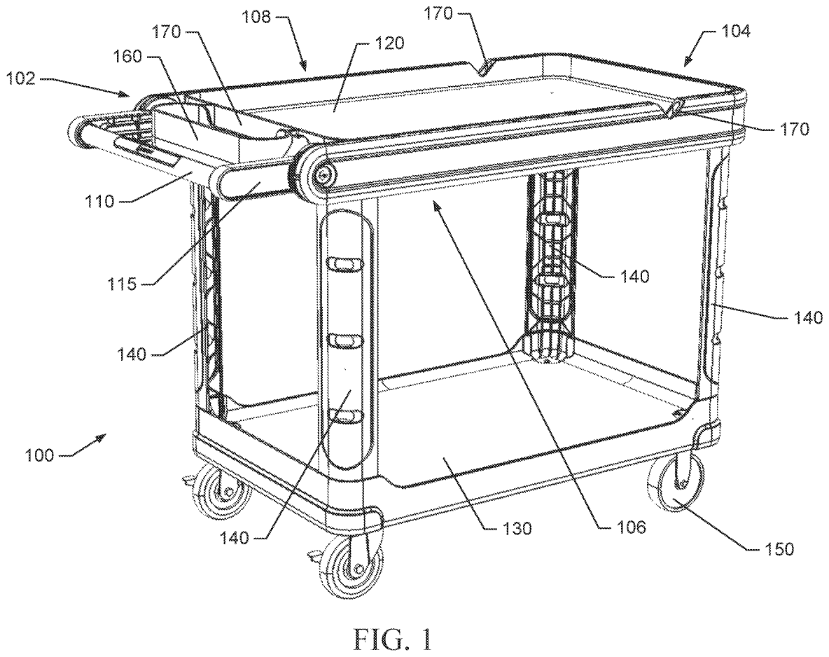

As indicated above, some example embodiments may relate to the provision of a utility cart 100 with an improved design. Referring now to FIG. 1, the utility cart 100 may include a handle 110, a top support platform 120, a bottom support platform 130, upright support members 140 and a mobility assembly. In some embodiments, the handle 110 may be a bar that may be disposed a distance away from the utility cart and may be operably coupled to the utility cart 100 at the top support platform 120 to allow for a user of the utility cart 100 to apply forces to move and steer the utility cart 100. When viewed from above, the utility cart 100 may have a substantially rectangular shape. In this regard, the utility cart 100 may have a first end 102 and a second end 104 and a first side 106 and a second side 108. The first end 102 and the second end 104 may both be equal to each other in length and shorter than each of the first side 106 and second side 108 of the utility cart 100. Thus, in some embodiments, the handle 110 may be operably coupled to the utility cart 100 at the first end 102 of the utility cart 100 proximate to the top support platform 120 so that the handle 110 may be at a comfortable position for the user of the utility cart 100 to interact with. The handle 110 of some example embodiments may also be adjustable. In this regard, the handle 110 may operably couple to the top support platform 120 via at least one extension arm 115. The at least one extension arm 115 may be pivotably operably coupled to the top support platform 120 such that an angle formed between the at least one extension arm 115 and the top support platform 120 may be adjustable. The handle 110 may therefore be fixed in whatever angle or position the user may desire via operable coupling of the at least one extension arm 115 to the top support platform 120.

The top support platform 120 may be disposed a distance above the bottom support platform 130 and supported by the upright support members 140. In some embodiments, there may be four total upright support members 140 supporting the top support platform 120, with one upright support member 140 disposed at each corner of the utility cart 100. It should be noted that in some other embodiments, more or fewer upright support members 140 may be used to support the top support platform 120. The top and bottom support platforms (120, 130) may both be planar surfaces substantially parallel to each other that may be capable of supporting objects thereon. In some cases, the top support platform 120 and the bottom support platform 130 may each include a lip that extends around the entire perimeter of each of the top and bottom support platforms (120, 130) that may help retain objects on respective support platforms by prohibiting the objects from rolling off the utility cart 100.

In some cases, some parts of the utility cart 100 may be formed from structural foam. In this regard, the top and bottom support platforms (120, 130), the handle 110, and some of the accessories described below may be formed from the structural foam. In some embodiments, the structural foam may be a black or gray color which may be a traditional appearance for many structural foams. The use of structural foam to construct the cart 100 may allow the cart 100 to achieve a higher stiffness-to-weight ratio than standard injection molded utility carts while also being more cost effective to produce. In some cases, products formed from structural foam may achieve a weight reduction of approximately 10-20% compared to their injection molded counterparts. However, the utility cart 100 of some embodiments may also include portions that are formed via an injection molding process. In this regard, the upright support members 140 may be injection molded from impact modified polypropylene, which may allow for the upright support members 140 to be colored without impacting the structural integrity of the cart 100.

Operably coupled to an underside of the bottom support platform 130 may be the mobility assembly, which may include a plurality of wheels 150. In some cases, the utility cart may include a total of four wheels 150, though more or less may also be used in other cases. The wheels 150 may be disposed at each of the four corners of the utility cart 100 shown in FIG. 1 in order to maximize the balance of the utility cart 100 and limit instability of the cart 100. In an example embodiment, all of the wheels 150 on the utility cart may be caster wheels. In such cases, all of the wheels 150 may be capable of turning 360° to allow the utility cart 100 to travel in any direction regardless of the orientation of the cart 100. In some other cases, the two wheels 150 disposed at the first end 102 may be caster wheels and the two wheels disposed at the second end 104 may be fixed wheels. In this regard, the utility cart 100 may be capable of being steered from the handle 110 at the first end 102 responsive to the caster wheels being capable of turning in any direction. In another example embodiment, the two wheels 150 disposed at the second end 104 may be caster wheels and the two wheels disposed at the first end 102 may be fixed wheels. In this regard, the utility cart 100 may be capable of being steered at the second end 104 via the handle 110 responsive to the caster wheels being capable of turning in any direction. In some cases, the wheels 150 may be fitted with a parking brake on each wheel 150. Each parking brake may immobilize the respective wheel 150 to which it may be operably coupled. With the parking brake for each wheel 150 engaged, the utility cart 100 may be held in place and may not roll anywhere. This feature may be useful when work may be done on the utility cart 100 itself, or in other cases where the cart 100 may roll away from the user unintentionally.

In some embodiments, the utility cart 100 may further include a storage pocket 160. The storage pocket 160 may be disposed between the handle 110 and the top support platform 120 in order to be easily accessible by the user of the utility cart 100. The storage pocket 160 may be capable of storing a number of objects of many different varieties. For example, the storage pocket 160 may store hand tools, writing utensils, or other miscellaneous objects of similar sizes. The storage pocket 160 may be disposed a distance away from the handle 110 due to the extension arms 115 extending away from the top support platform 120. In some cases, the distance between the handle 110 and the storage pocket 160 may vary since the position of the handle 110 may be adjustable. The storage pocket 160 may be disposed between two extension arms 115 of the handle 110. In this regard, there may be excess space on either longitudinal end of the storage pocket 160 between the storage pocket 160 and the extension arms 115. This space may provide some buffer between the storage pocket 160 and the extension arms 115 so that any objects stored in the storage pocket 160 do not interfere with the adjustment of the position of the handle 110 via the extension arms 115. In some cases, the space between the longitudinal ends of the storage pocket 160 and the extension arms 115 may account for approximately 10% of the total length of the handle 110, or a total distance between the two extension arms 115. In other words, the storage pocket 160 may have a length that is approximately 90% of the total length of the handle 110, or the total distance between the two extension arms 115.

The utility cart 100 may further include a technology receptacle 170 disposed between the storage pocket 160 and the top support platform 120. The technology receptacle 170 may be designed to support a personal electronic device, such as a mobile phone device or a tablet device, in a position where the personal electronic device may be used or viewed by the user of the utility cart 100. As such, the technology receptacle 170 may keep the personal electronic device separate from the storage of other objects within the storage pocket 160 to prevent any interference of the personal electronic device due to objects in the storage pocket 160. In contrast with the technology receptacle 170, the storage pocket 160 may be designed to hold a variety of tools, materials and objects that the user may want to keep separate or more accessible than objects in either of the top or bottom support platforms (120, 130). In an example embodiment, the distance between the storage pocket 160 and the handle 110 may not exceed a width of the storage pocket 160 measured between a shared wall with the technology receptacle 170 and a front wall of the storage pocket 160 closest to the handle 110. In some other cases, the distance between the storage pocket 160 and the handle 110 may be within a range of approximately 50-75% of the width of the storage pocket 160. This may ensure that there is sufficient space between the storage pocket 160 and the handle so that they may not interfere with each other as the user may interact with the handle 110 or the storage pocket 160. In an example embodiment, the technology receptacle 170 may have an overall length that is approximately 75% of the length of the storage pocket 160. In other words, the overall length of the technology receptacle 170 may be approximately 50-60% of the total length of the handle 110, or the total distance between the two extension arms 115. In an example embodiment, the top support platform 120 may be wider than the handle 110 is long. In other words, the length of the handle 110 may be approximately 90% of a width of the top support platform 120. Similarly, the length of the technology receptacle 170 may be approximately 40-50% of the width of the top support platform 120.

Thus, the technology receptacle 170 may hold the user's personal electronic device therein. For example, in a scenario where the utility cart 100 may be used for order fulfillment situations, the personal electronic device may contain a list of items needed to fulfill a particular order. Thus, the user of the cart 100 may need to easily see the screen of the personal electronic device being supported in the technology receptacle 170. In this regard, the technology receptacle 170 may provide stability of the personal electronic device disposed in the technology receptacle 170 so that the movement of the utility cart 100 does not knock over the personal electronic device. Additionally, the technology receptacle 170 may be designed to provide the user with an ergonomic view of the display of the personal electronic device so that the user of the utility cart 100 does not need to strain to view the device.

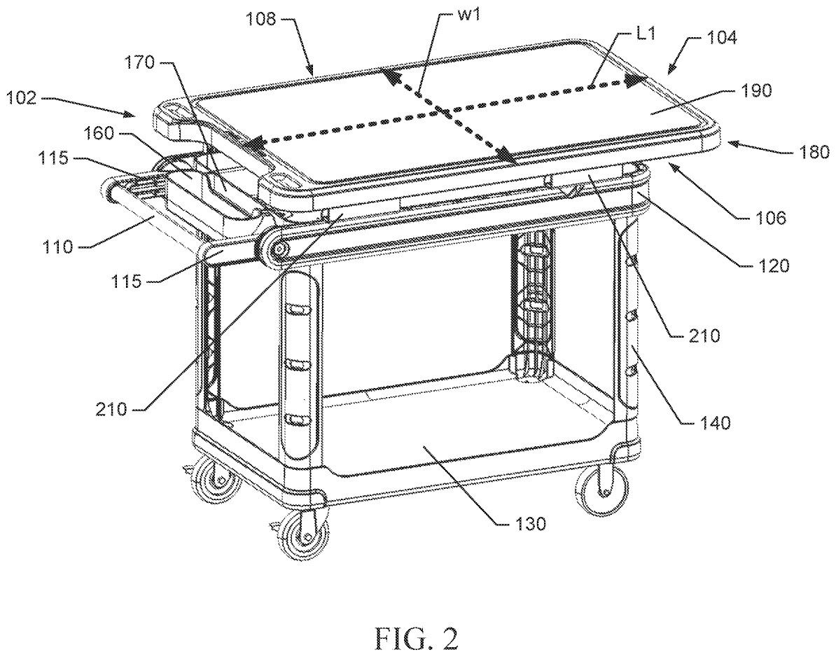

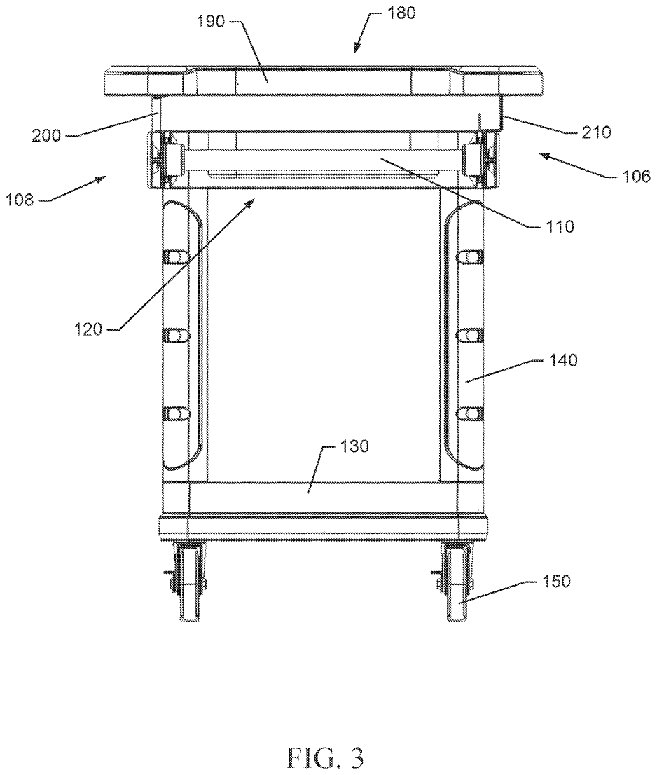

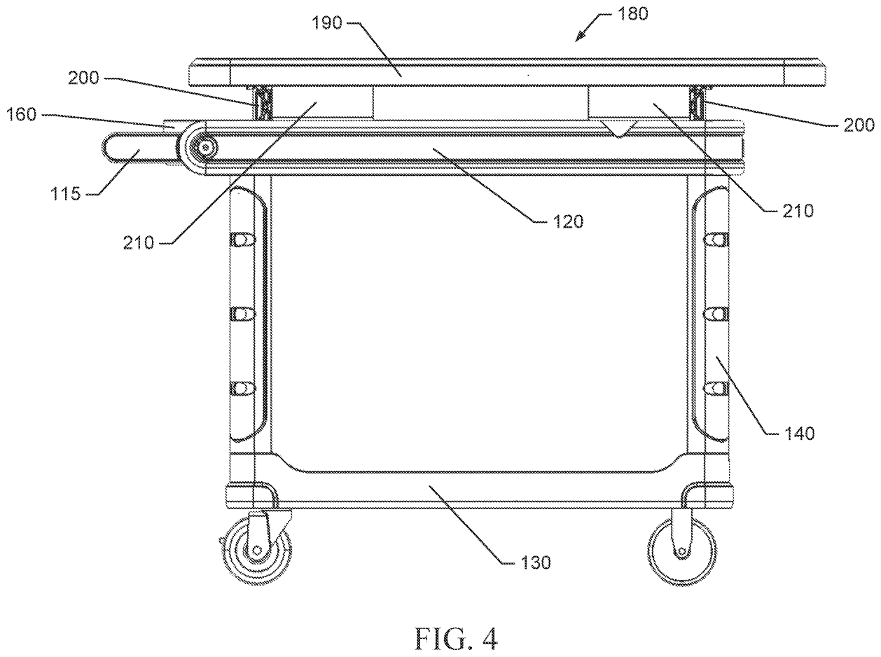

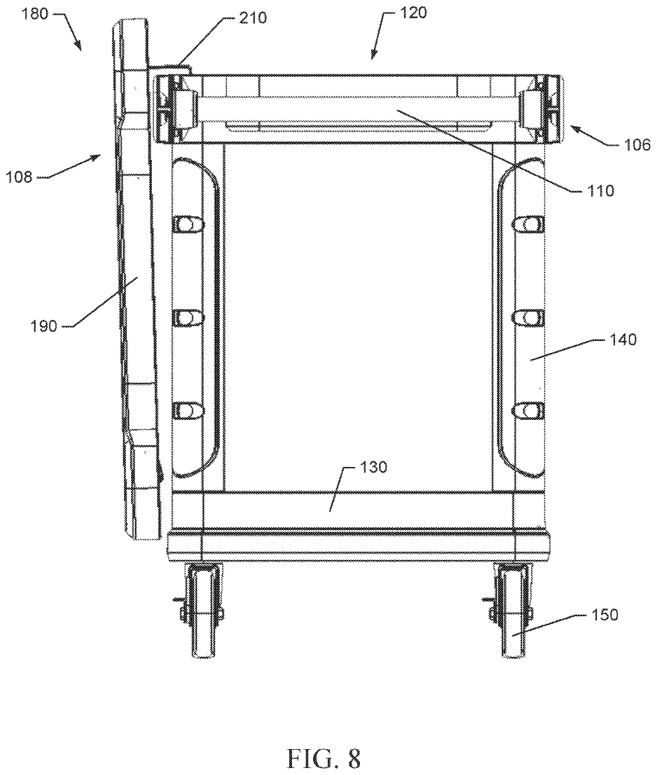

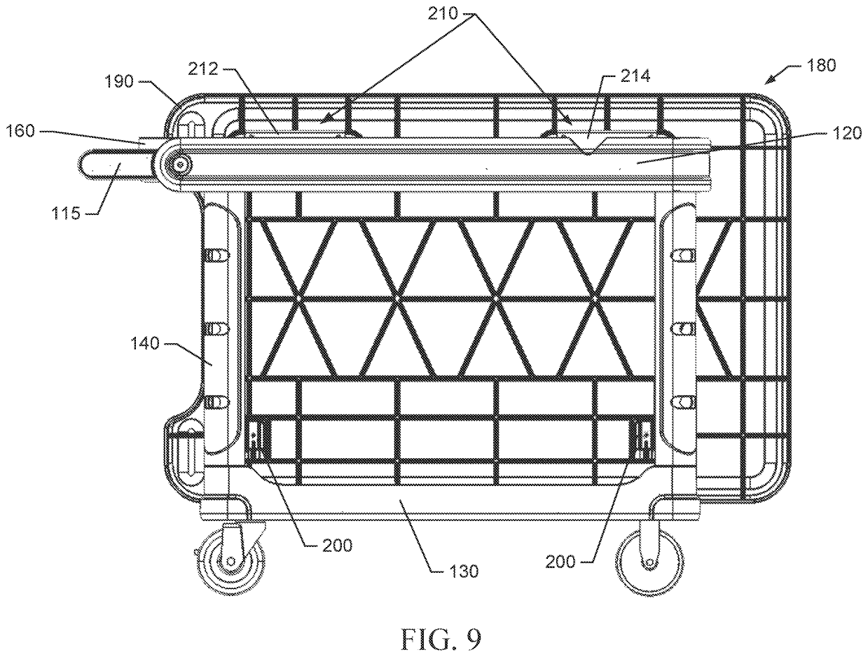

FIGS. 2-9 depict various views of the utility cart 100 having a work surface 180 in accordance with an example embodiment. In this regard, the utility cart 100 of some embodiments may include a work surface 180 that may be both removably and hingedly operably coupled to the top support platform 120. The work surface 180 may provide an enlarged planar surface for the user to utilize for various work tasks that may require more space to operate than may be provided by the top support platform 120. Thus, the work surface 180 may have a larger area than the top support platform 120. As shown in FIGS. 2-4, the work surface 180 may overhang the top support platform 120 on the first and second sides (106, 108) of the top support platform 120 by about 10-15% of the width (w1) of the work surface 180. In some cases, the work surface 180 may overhang the second end 104 of the top support platform 120 by about 10-15% of the length (L1) of the work surface 180. In fact, in some cases, the work surface 180 may have a total area approximately 1.5 times larger than an area of the top support platform 120. The work surface 180, in some cases, may include a lip that may extend around an entire perimeter of the work surface 180 which may help retain objects on the work surface 180 as the utility cart 100 moves and/or the work surface 180 is in use. In an example embodiment, the work surface 180 may further include a groove or a channel that may extend around an entire perimeter of the work surface 180 just inside of the lip. In this regard, the groove or channel may also help retain objects on the work surface 180 as the utility cart 100 moves and/or the work surface 180 is in use.

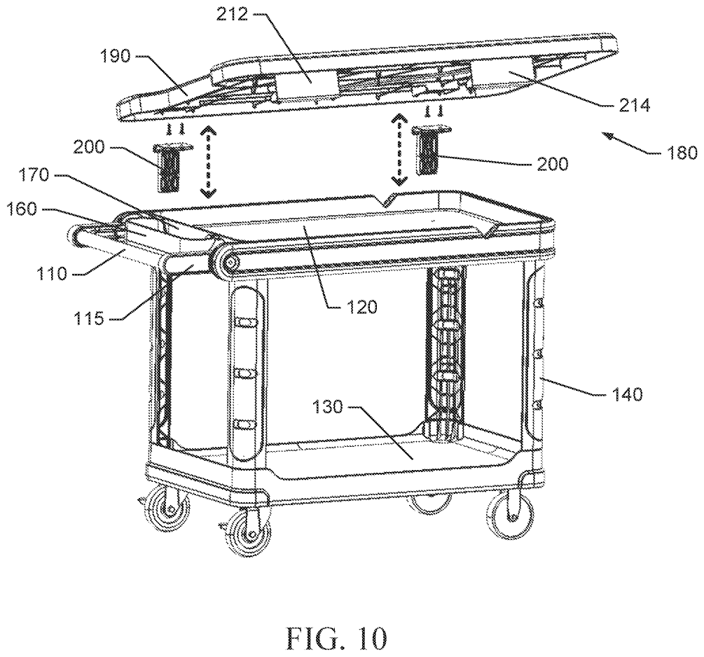

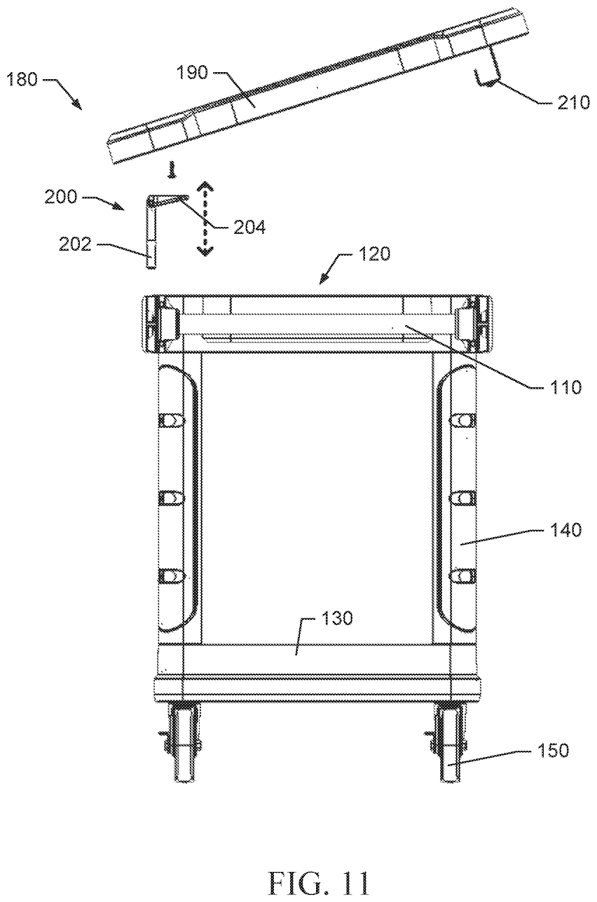

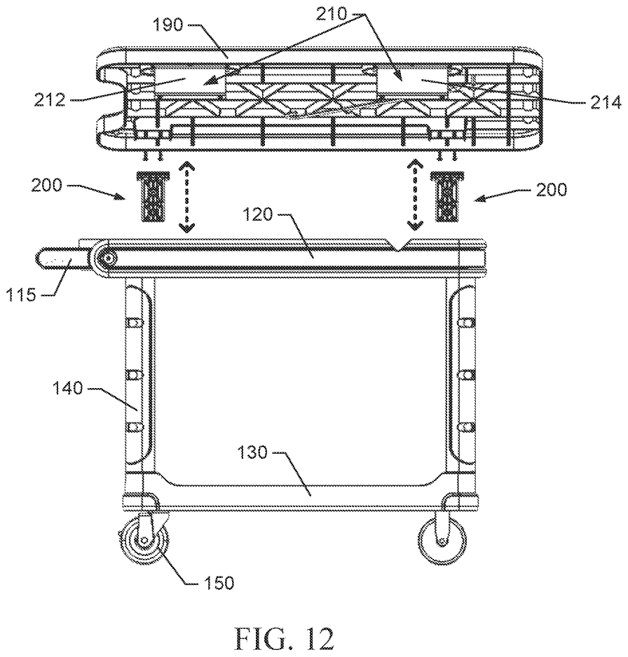

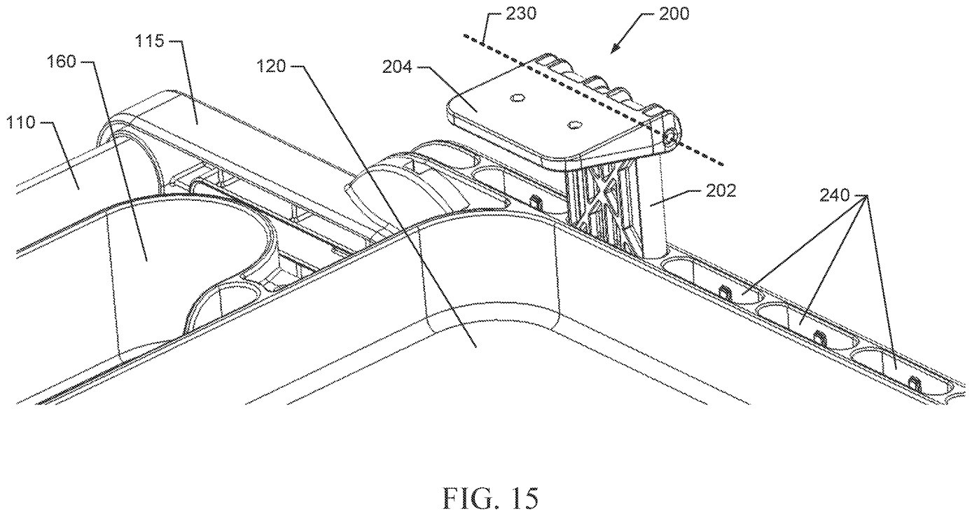

In an example embodiment, the work surface 180 may include a planar member 190, a hinge assembly 200, a hook assembly 210, and a prop rod 220. In this regard, the lip and the groove or channel discussed above may be part of the planar member 190. The planar member 190 may have a larger area than the top support platform 120, which in some cases may be approximately 1.5 times larger to approximately 2 times larger. The planar member 190 may be removably operably coupled, and hingedly operably coupled, to the top support platform 120 via the hinge assembly 200, as will be described later in reference to FIGS. 10-12 and 15. The hinge assembly 200 may also define a pivot axis 230 there through, about which the planar member 190 may pivot. The hinge assembly 200 may operably couple to the planar member 190 proximate to an edge disposed at an opposite side of the planar member 190 from the hook assembly 210, but both the hinge assembly 200 and the hook assembly 210 may operably couple to the planar member 190 at an underside thereof such that the hinge assembly 200 and the hook assembly 210 may interface with the top support platform 120 of the utility cart 100. In some cases, the hinge assembly 200 and the hook assembly 210 may be referred to as the first and second support assemblies, respectively. Responsive to the planar member 190 pivoting about the pivot axis 230, the hook assembly 210 may be disposed above (i.e. further away from the top support platform 120 than) the hinge assembly 200. In some cases, both the hinge assembly 200 and the hook assembly 210 may be operably coupled to the planar member 190 via a fastener or a plurality of fasteners and in other cases via other securing means.

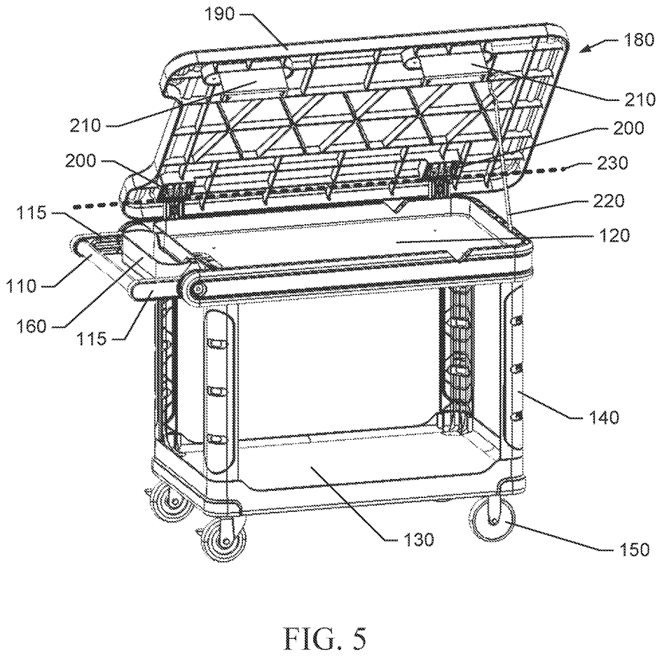

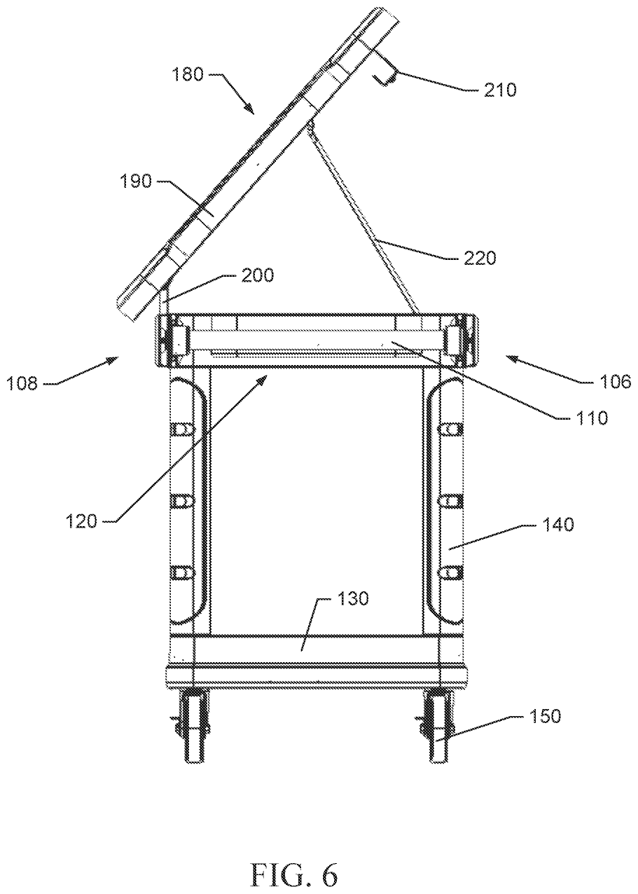

As shown in FIGS. 2-6, the planar member 190 may be pivotable about the pivot axis 230 between a first position (shown in FIGS. 2-4) in which the planar member 190 may be disposed parallel to the top support platform 120, and a second position (shown in FIGS. 5 and 6) in which the planar member 190 may not be parallel with the top support platform 120. In this regard, the first position may be a use position. In the use position, the user may place various objects on the work surface 180 to perform various tasks that may require a larger planar area to work on than the top support platform 120 could provide. Thus, in the first position, the top support platform 120 may be covered by the work surface 180. On the other hand, the second position may be an open position. In this regard, any objects stored within the top support platform 120 that may need to be accessed quickly or without entirely removing the work surface 180, the user may pivot the planar member 190 from the first position into the second position about the pivot axis 230 via the hinge assembly 200 in order to quickly and easily gain more access to the top support platform 120 without the work surface 180 in the way. In the second position, the prop rod 220 may be utilized to hold the planar member 190 in the second position. In this regard, the prop rod 220 may be operably coupled to an underside of the planar member 190 as well. One end of the prop rod 220 may be operably coupled to the planar member 190 with a ball and socket type joint, which may give the opposite end of the prop rod 220 a wide range of motion and possible positions. Thus, the first end of the prop rod 220 may remain coupled to the planar member 190 while the second end of the prop rod 220 may be moved by the user down to interface with the top support platform 120 such that the prop rod 220 may support the weight of the planar member 190 in the second position. Accordingly, the second position may not be defined as forming a specific angle between the top support platform 120 and the planar member 190. In other words, due to the adjustability of the prop rod 220, the planar member 190 may form a range of angles with the top support platform 120 depending on the placement of the second end of the prop rod 220 and depending on the needs of the user. In an example embodiment, the planar member 190 may be made from High Density Polyethylene (HDPE) structural foam.

In any case, while the work surface 180 may be in the first position, the hook assembly 210 may rest on the lip of the top support platform 120 at an opposite side of the utility cart 100 from where the hinge assembly 200 operably couples to the utility cart 100 to support the planar member 190 substantially parallel to the top support platform 120. In some cases, this may also support the planar member 190 at a specified distance above the top support platform 120. Also in the first position, the prop rod 220 may be operably coupled to the underside of the planar member 190 at both the first end and the second end of the prop rod 220. On the contrary, in the second position, the hook assembly 210 may be disposed a distance above the hinge assembly 200, and the prop rod 220 may be operably coupled to the underside of the planar member 190 at the first end and the second end of the prop rod may be operably coupled to the top support platform 120. In some cases, the prop rod 220 may be a 5/16 inch diameter rod made from 1045 steel.

As shown in the example embodiment depicted in FIGS. 10-12 and 15, the hinge assembly 200 of the work surface 180 may be removably operably coupled to the top support platform 120. In this regard, the lip of the top support platform 120 may include a plurality of accessory slots 240. The plurality of accessory slots 240 of some cases may be disposed around the entire perimeter of the top support platform 120. The plurality of accessory slots 240 may operably couple to a variety of accessories that may be used with the utility cart 100. In some cases, the plurality of accessory slots 240 may extend through an entire depth of the lip of the top support platform 120 such that the accessory slots 240 may allow objects to pass through the top support platform 120. In an example embodiment, the plurality of accessory slots 240 may include a locking structure disposed therein that may be configured to operably couple with a complementary locking structure disposed on the accessory disposed therein. In the present case, the hinge assembly 200 may include a first hinge member 202 which may removably operably couple to a respective one of the accessory slots 240 disposed at the top support platform 120 and a second hinge member 204 which may operably couple to the underside of the planar member 190. The first hinge member 202 and the second hinge member 204 may be pivotably operably coupled to one another at the pivot axis 230. This may allow the planar member 190 to pivot relative to the top support platform 120 between the first and second positions. As such, with the planar member 190 in the first position, the first hinge member 202 may form an angle of approximately 90° with the second hinge member 204 and thus also with the planar member 190. In an example embodiment, the hinge assembly 200 may include any number of hinge assemblies, from as few as one hinge assembly 200 to as many that may fit. In the example embodiments shown and described herein, the work surface 180 may include two hinge assemblies 200.

As shown in FIGS. 10-12, the hinge assembly 200 may be installed into the accessory slots 240 by sliding the first hinge member 202 in a direction going towards the top support platform 120 and substantially perpendicular to the top support platform 120. Therefore, the hinge assembly 200 may be removed from the accessory slots 240 by sliding the first hinge member 202 in a direction substantially normal to the top support platform 120. In some cases, responsive to the first hinge member 202 sliding out of the respective accessory slot 240, the work surface 180 may be removed from the utility cart 100. In an example embodiment, the hinge assembly 200 may be formed from nylon 6/6 PA+50% GF.

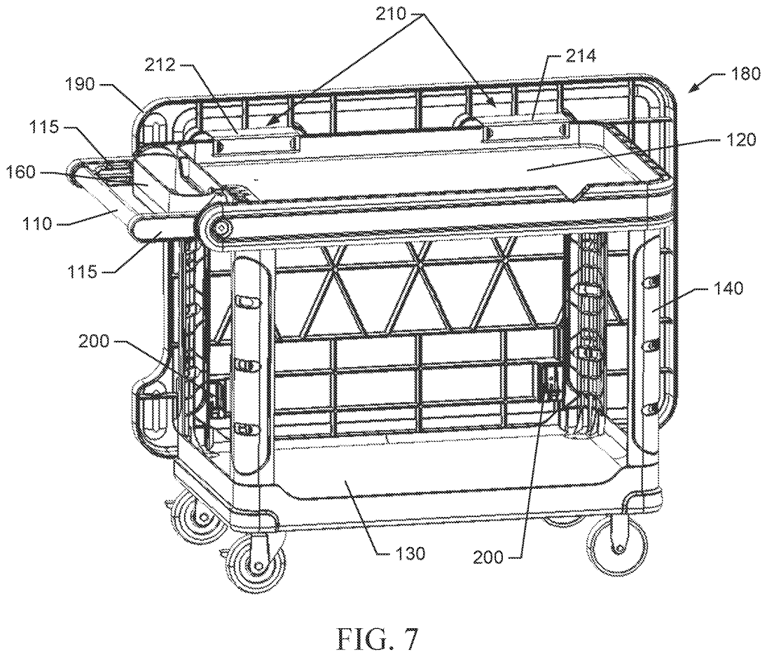

As shown in FIGS. 7-9 and 16, responsive to the work surface 180 being removed from the utility cart 100, the work surface 180 may be placed into a third position which may be a storage position. In the storage position, the work surface 180 may be supported by the hook assembly 210. In this regard, the hook assembly 210 may operably couple with the lip of the top support platform 120 such that the planar member 190 may be disposed at either of the first or second sides (106, 108) of the utility cart 100. In other words, the work surface 180 may be hung from the first side 106 or second side 108 of the utility cart 100 by the hook assembly 210. In some cases, the hook assembly 210 may include a first hook 221 and a second hook 214. However, in an example embodiment, the hinge assembly 200 may include any number of hooks from as few as one to as many that may fit. In some cases, the hooks in the hook assembly 210 may be substantially C shaped but with planar sides and approximately 90° angles. In this regard, the top side of the C shape may operably couple to the underside of the planar member 190 via a fastener or fasteners, or in other cases, by any other securing means. The upright side of the C shape may thus extend perpendicular to the top side of the C shape. In an example embodiment, the upright side of the C shaped hook may extend perpendicularly to the planar member 190. The bottom side of the C shape may extend perpendicular to the upright side, but parallel to the top side of the C shape. Thus, with the planar member 190 in the first position, parallel to the top support platform 120, the work surface 180 may rest the bottom side of the C shape of the hooks in the hook assembly 210 on the lip of the top support platform 120. On the other hand, in the third position, the planar member 190 may be disposed substantially perpendicularly to the top support platform 120 and be supported by the hook assembly 210. In this regard, the upright side of the C shaped hooks (212, 214) may rest on the lip of the top support platform 120. In other words, the lip of the top support platform 120 may fit into the open center area inside the middle of the C shaped hooks (212, 214) so that the hooks (212, 214) may grab onto the lip via the C shape.

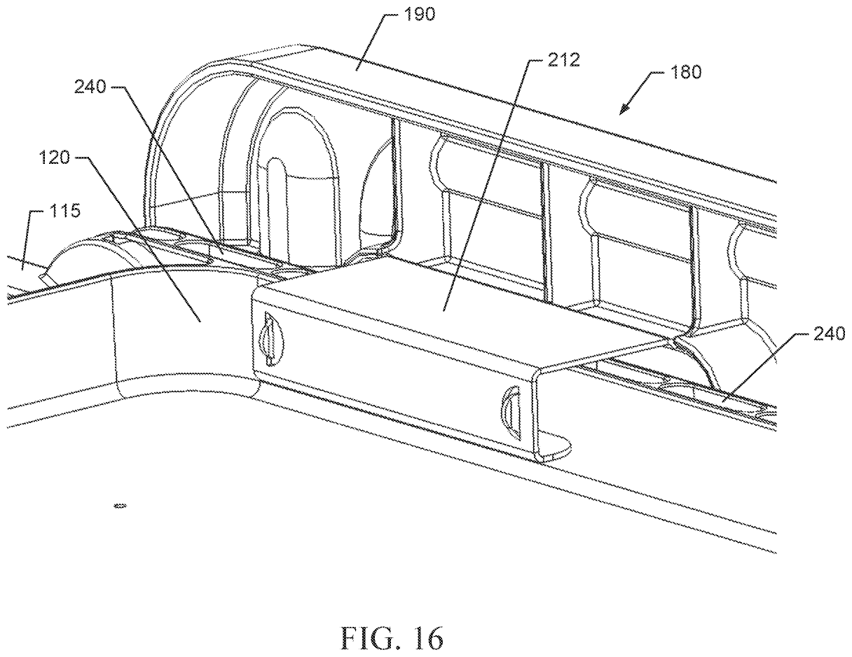

In some cases, the hooks (212, 214) of the hook assembly 210 may be substantially G shaped instead of C shaped. In this regard, the same sides of the C shaped hooks (212, 214) may be present but the G shaped hooks (212, 214) may include an additional upright side disposed perpendicular to the top and bottom sides and parallel to the upright side. This additional side may extend for only a fraction of the length of the upright side and may help provide a secure fit for the hooks (212, 214) onto the lip of the top support platform 120 when the work surface 180 is in the third position, as shown in FIG. 16. In this regard, the distance between the end of the additional side and the top side may be substantially equal to the width of the lip of the top support platform 120 so that there is minimal play between the hook assembly 210 and the lip of the top support platform 120 when the work surface 180 is in the third position. In example embodiments where the hooks (212, 214) are C shaped, the distance between the bottom side and the top side may be substantially equal to the width of the lip of the top support platform 120 so that there is minimal play between the hook assembly 210 and the lip of the top support platform 120 when the work surface 180 is in the third position. In an example embodiment, the first and second hooks (212, 214) may be made from 12 gauge sheet metal and may be bent into their respective shape.

In some example embodiments, the utility cart 100 may include an accessory support assembly 250. In this regard, the accessory support assembly 250 may include the handle 110, the extension arms 115, the top support platform 120, and work surface 180. The accessory support assembly 250 may be configured to retain additional accessories for the utility cart 100 therein or otherwise operably couple to additional accessories for the utility cart 100.

Some example embodiments may provide for a utility cart. The utility cart may include a handle, a top support platform, to which the handle may be operably coupled, a bottom support platform which may be parallel to the top support platform, at least one upright support member which may operably couple the bottom support platform to the top support platform, a mobility assembly which may be operably coupled to the bottom support platform, and a work surface that may be alternatingly removably operably coupled to the top support platform via a first support assembly and a second support assembly. The first support assembly and the second support assembly may operably couple the work surface to the top support platform in different orientations.

The utility cart of some embodiments may include additional, optional features, and/or the features described above may be modified or augmented. Some examples of modifications, optional features and augmentations are described below. It should be appreciated that the modifications, optional features and augmentations listed below may each be added alone, or they may be added cumulatively in any desirable combination. For example, in some embodiments, the work surface may include a planar member which may have a larger area than the top support platform,, and a prop rod which may be disposed on an underside of the planar member. In some cases, the first support assembly may be a hinge assembly which may operably couple the planar member to the top support platform and may define a pivot axis there through. In an example embodiment, the second support assembly may be a hook assembly which may operably couple the planar member to the top support platform at an opposite edge of the planar member from the hinge assembly. In some cases, the planar member may be pivotable about the pivot axis between a first position in which the planar member may be disposed parallel to the top support platform and a second position in which the planar member may not be parallel with the top support platform. In an example embodiment, in the first position, the hook assembly may rest on a lip of the top support platform at an opposite side of the utility cart from the hinge assembly to support the planar member. In some cases, in the second position, the hook assembly may be pivoted away from the top support platform. In an example embodiment, when the planar member may be in the first position, the prop rod may be operably coupled to the underside of the planar member at a first end and a second end of the prop rod. In some cases, when the planar member may be in the second position, the prop rod may be operably coupled to the underside of the planar member at the first end of the prop rod and the second end of the prop rod may be operably coupled to the top support platform. In an example embodiment, when the planar member may be removably operably coupled to the top support platform via the hinge assembly, the hinge assembly may include a first hinge member removably operably coupled to an accessory slot disposed at the top support platform and a second hinge member operably coupled to the underside of the planar member. In an example embodiment, the first hinge member and the second hinge member may be pivotably operably coupled to one another at the pivot axis. In some cases, responsive to the first hinge member sliding out of the accessory slot, the work surface may be removed from the utility cart. In an example embodiment, when the planar member may be removably operably coupled to the top support platform via the hook assembly, the hook assembly may operably couple with a lip of the top support platform such that the planar member may be disposed at a side of the utility cart in a storage position. In some cases, in the storage position, the planar member may be substantially perpendicular to the top support platform.

Some example embodiments may provide for an accessory support assembly for a utility cart. The accessory support assembly may include a handle, a top support platform to which the handle may be operably coupled, the top support platform may include a plurality of accessory slots which may be disposed around a perimeter thereof, at least one extension arm which may operably couple the handle to the top support platform, and a work surface that may be alternatingly removably operably coupled to the top support platform via a first support assembly and a second support assembly. The first support assembly and the second support assembly may operably couple the work surface to the top support platform in different orientations.

Some example embodiments may provide for a work surface for a utility cart. The work surface may include a planar member which may have a larger area than a top support platform of the utility cart, and a prop rod which may be disposed on an underside of the planar member. The work surface may be alternatingly removably operably coupled to the top support platform via a first support assembly and a second support assembly. The first support assembly may be a hinge assembly which may operably couple the planar member to the top support platform and may define a pivot axis there through. The second support assembly may be a hook assembly which may operably couple the planar member to the top support platform at an opposite edge of the planar member from the hinge assembly.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

That which is claimed:

1. A utility cart comprising:
a handle;
a top support platform, to which the handle is operably coupled;
a bottom support platform disposed parallel to the top support platform;
a plurality of upright support members operably coupling the bottom support platform to the top support platform;
a mobility assembly operably coupled to the bottom support platform; and
a work surface alternatingly removably operably coupled to the top support platform via a first support assembly and a second support assembly,
wherein the first support assembly and the second support assembly operably couple the work surface to the top support platform in different orientations.

2. The utility cart of claim 1, wherein the work surface comprises:
a planar member having a larger area than the top support platform; and
a prop rod disposed on an underside of the planar member,
wherein the first support assembly is a hinge assembly operably coupling the planar member to the top support platform and defining a pivot axis there through, and
wherein the second support assembly is a hook assembly operably coupling the planar member to the top support platform at an opposite edge of the planar member from the hinge assembly.

3. The utility cart of claim 2, wherein the planar member is pivotable about the pivot axis between a first position in which the planar member is disposed parallel to the top support platform and a second position in which the planar member is not parallel with the top support platform.

4. The utility cart of claim 3, wherein in the first position, the hook assembly rests on a lip of the top support platform at an opposite side of the utility cart from the hinge assembly to support the planar member.

5. The utility cart of claim 3, wherein in the second position, the hook assembly is pivoted away from the top support platform.

6. The utility cart of claim 3, wherein when the planar member is in the first position, the prop rod is operably coupled to the underside of the planar member at a first end and a second end of the prop rod, and
wherein when the planar member is in the second position, the prop rod is operably coupled to the underside of the planar member at the first end of the prop rod and the second end of the prop rod is operably coupled to the top support platform.

7. The utility cart of claim 2, wherein when the planar member is removably operably coupled to the top support platform via the hinge assembly, the hinge assembly comprises a first hinge member removably operably coupled to an accessory slot disposed at the top support platform and a second hinge member operably coupled to the underside of the planar member, and
wherein the first hinge member and the second hinge member are pivotably operably coupled to one another at the pivot axis.

8. The utility cart of claim 7, wherein responsive to the first hinge member sliding out of the accessory slot, the work surface is removed from the utility cart.

9. The utility cart of claim 2, wherein when the planar member is removably operably coupled to the top support platform via the hook assembly, the hook assembly operably couples with a lip of the top support platform such that the planar member is disposed at a side of the utility cart in a storage position.

10. The utility cart of claim 9, wherein in the storage position, the planar member is substantially perpendicular to the top support platform.

11. An accessory support assembly for a utility cart, the accessory support assembly comprising:
a handle;
a top support platform, to which the handle is operably coupled, the top support platform comprising a plurality of accessory slots disposed around a perimeter thereof;
at least one extension arm which operably couples the handle to the top support platform; and
a work surface alternatingly removably operably coupled to the top support platform via a first support assembly and a second support assembly,
wherein the first support assembly and the second support assembly operably couple the work surface to the top support platform in different orientations.

12. The accessory support assembly of claim 11, wherein the work surface comprises:
a planar member having a larger area than the top support platform; and
a prop rod disposed on an underside of the planar member
wherein the first support assembly is a hinge assembly operably coupling the planar member to the top support platform and defining a pivot axis there through;
wherein the second support assembly is a hook assembly operably coupling the planar member to the top support platform at an opposite edge of the planar member from the hinge assembly.

13. The accessory support assembly of claim 12, wherein the planar member is pivotable about the pivot axis between a first position in which the planar member is disposed parallel to the top support platform and a second position in which the planar member is not parallel with the top support platform.

14. The accessory support assembly of claim 12, wherein when the planar member is removably operably coupled to the top support platform via the hinge assembly the hinge assembly comprises a first hinge member removably operably coupled to an accessory slot of the plurality of accessory slots disposed at the top support platform and a second hinge member operably coupled to the underside of the planar member, and
wherein the first hinge member and the second hinge member are pivotably operably coupled to one another at the pivot axis.

15. The accessory support assembly of claim 12, wherein when the planar member is removably operably coupled to the top support platform via the hook assembly, the hook assembly operably couples with a lip of the top support platform such that the planar member is disposed at a side of the utility cart in a storage position.

16. The accessory support assembly of claim 15, wherein in the storage position, the planar member is substantially perpendicular to the top support platform.

17. A work surface for a utility cart, the work surface comprising:
a planar member having a larger area than a top support platform of the utility cart; and
a prop rod disposed on an underside of the planar member,
wherein the work surface is alternatingly removably operably coupled to the top support platform via a first support assembly and a second support assembly,
wherein the first support assembly is a hinge assembly operably coupling the planar member to the top support platform and defining a pivot axis there through;
wherein the second support assembly is a hook assembly operably coupling the planar member to the top support platform at an opposite edge of the planar member from the hinge assembly.

18. The work surface of claim 17, wherein the planar member is pivotable about the pivot axis between a first position in which the planar member is disposed parallel to the top support platform and a second position in which the planar member is not parallel with the top support platform.

19. The work surface of claim 17, wherein when the planar member is removably operably coupled to the top support platform via the hinge assembly the hinge assembly comprises a first hinge member removably operably coupled to an accessory slot disposed at the top support platform and a second hinge member operably coupled to the underside of the planar member, and
wherein the first hinge member and the second hinge member are pivotably operably coupled to one another at the pivot axis.

20. The work surface of claim 17, wherein when the planar member is removably operably coupled to the top support platform via the hook assembly, the hook assembly operably couples with a lip of the top support platform such that the planar member is disposed at a side of the utility cart in a storage position, and
wherein in the storage position, the planar member is substantially perpendicular to the top support platform.
